(19) United States
(12) Patent Application Publication
(10) Pub. No.: US 2008/0056440 A1
(43) Pub. Date: Mar. 6, 2008

(54) METHOD AND DEVICE FOR SUPPORTING AN IMAGE-AIDED MEDICAL PROCEDURE

(76) Inventors: Daniel Fischer, Erlangen (DE); Jasmina Orman, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/839,731

(22) Filed: Aug. 16, 2007

(30) Foreign Application Priority Data
Sep. 4, 2006 (DE) 10 2006 041 457.8

(51) Int. Cl. A61B 6/03; G06K 9/36

(52) U.S. Cl. 378/21; 378/37; 382/128

(57) ABSTRACT

In a method and a device for supporting the implementation of a medical procedure on a patient, the progress of the procedure is visibly indicated for the patient with an optical display. The stress associated with the procedure is thereby subjectively reduced for the patient.

### BACKGROUND OF THE INVENTION

1. Field of the Invention

The present invention concerns a method for supporting the implementation of an image-aided medical procedure on a patient. Moreover, the invention concerns a device for image-aided implementation of a medical procedure that operates according to such a method.

2. Description of the Prior Art

The implementation of a medical procedure is in many cases associated with a significant physical or psychological stress for the patient. The cause of this is that the patient finds himself or herself in an uncomfortable situation during a procedure that he or she can only observe and understand in a limited manner. Under the circumstances, the stress connected with this can lead to a reduced willingness of the patient to cooperate. This in turn can additionally increase the time required for the medical procedure.

Such a medical procedure can be, for example, a time-intensive x-ray examination in the framework of computed tomography or mammography in which a tomosynthesis (in which a 3D image data set (3D image) composed of a number of slice images is reconstructed from a number of individual 2D x-ray images), or it can be a procedure (for example a biopsy) that is supported with a tomographic or tomosynthetic x-ray image. The situation is particularly uncomfortable for female patients in mammography, in which the breast is held in a compressed state between a support plate and a compression plate during the x-ray acquisitions.

### SUMMARY OF THE INVENTION

An object of the present invention is to provide a method for supporting the implementation of an image-aided medical procedure on a patient, with which the stress subjectively perceived by the patient is reduced. A further object of the invention is to provide a device operating with such a method.

With regard to the method, the above object is achieved by a method according to the invention, wherein the implementation of an image-aided procedure on a patient is supported by the progress of the procedure being visibly, optically presented to the patient.

As used herein medical “procedure” means a procedure in which only one or a series of x-ray images is acquired and a procedure that, for example, occurs with the aid of x-ray images generated during the measure.

The invention is based on the insight that the subjective perception of the patient independent of the actual, objective stress can be influenced by external factors not directly connected with the procedure. The time duration of an uncomfortable situation is thus perceived to be less long when the patient can optically track the progress of the procedure.

The method according to the invention is particularly suitable for procedures in which a number of 2D x-ray images with different projection angles are acquired during the procedure or as a procedure, from which multiple 2D x-ray images a 3D image data set (in particular a tomosynthetic 3D image data set in mammography) is reconstructed.

Such a positive effect subjectively shortening the perceived duration of the measure for the patient is particularly achieved when the progress of the procedure is indicated in relation to a total duration. The patient thereby receives a conception of the temporal workflow, i.e. of the speed in which the procedure is implemented, and receives a conception of when the procedure will likely be ended.

This subjective time perception is moreover additionally shortened when the remaining duration of the procedure is recognizably indicated for the patient.

Such an optical display is also helpful for the operating personnel and also subjectively shortens for them the wait times between the beginning and the end of the procedure.

In a further embodiment of the invention, the progress of the procedure is additionally acoustically indicated. This is helpful for persons with weak eyesight.

The above object also is achieved in accordance with the present invention by a device that implements the method described above, including all variations and embodiments that are also described above.

### BRIEF DESCRIPTION OF THE DRAWINGS

FIG. 1 shows a device according to the invention using a tomosynthesis-capable mammography apparatus in a schematic representation,

FIG. 2 shows an exemplary embodiment illustrating how the progress of the measure in a mammography method is visualized.

### DESCRIPTION OF THE PREFERRED EMBODIMENTS

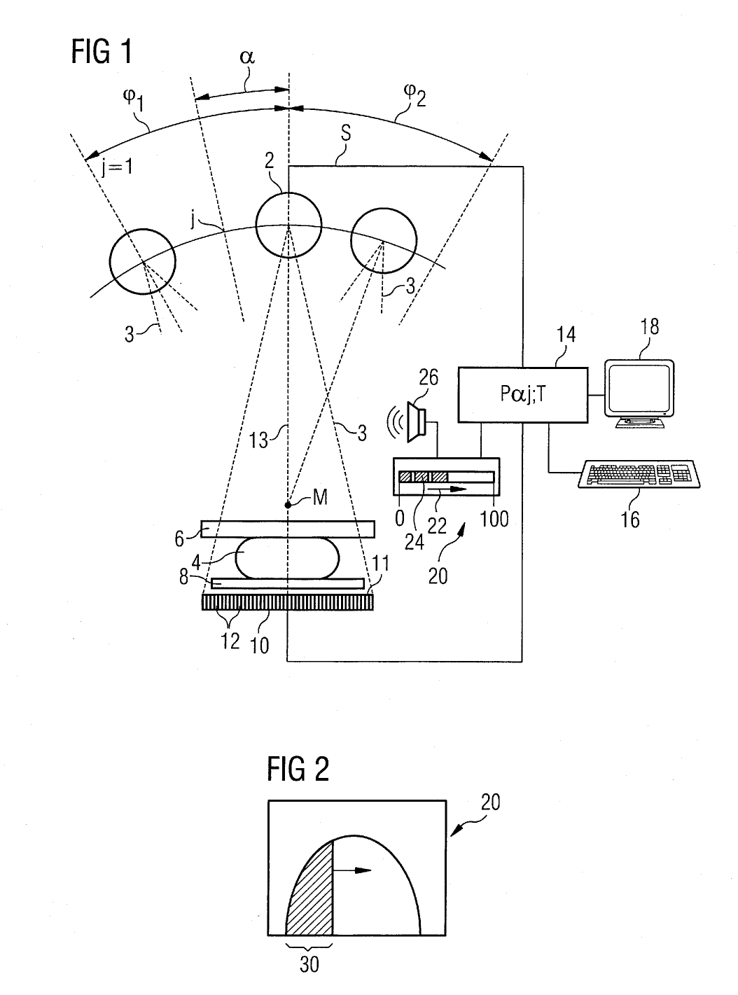

According to FIG. 1, the device has, for example, an imaging x-ray system (a tomosynthesis-capable mammography apparatus in the exemplary embodiment), including an x-ray tube 2 for generation of x-rays that pass through an examination subject 4. The examination subject 4 is a female breast that is composed between a compression plate 6 and a support plate 8. The x-rays 3 passing through the examination subject 4, the compression plate 6 and the support plate 8 are received by a large-surface digital x-ray detector 10 that is composed of a number of individual detectors 12 arranged in a matrix-like array, and whose acquisition surface 11 is parallel to the plates 6, 8.

The x-ray tube 2 is arranged so as to be positionally variable in a limited range relative to the examination subject and can be pivoted, for example, in different angle positions j=1 . . . n in a limited angle range φ1, φ2 around an axis M perpendicular to the plane of the drawing such that individual images of the examination subject 4 can be generated at projection angles αⱼ relative to the normal 13 of the acquisition surface 11 of the x-ray detector 10. The angle range (φ1, φ2 does not have to be symmetrical to the normal 13. These 2D x-ray images or the projection data P_{αj} respectively associated therewith are supplied to a control and evaluation device 14 (an image computer) that performs a reconstruction into a tomosynthetic 3D image data set composed of a number of slice images, shown on a monitor 18. The x-ray detector 10 is stationary during the pivot movement of the x-ray tube 2. In principle it is also possible to mutually pivot the x-ray detector 10 as well or to linearly displace the x-ray detector 10 following the pivot movement of the x-ray tube 2.

A movement of the x-ray tube 2 on a limited, linear track instead of the pivot is also allowable so that the height difference between x-ray detector 10 and x-ray tube remains constant. This linear track likewise does not necessarily have to proceed symmetrical to the normal 1. Given this linear movement an alignment of the x-ray tube 2 on the examination subject 4 ensues so that in this case individual images of the examination subject 4 are also acquired from different projection angles αⱼ, however in a limited angle range.

The control of the angle position j or (in the case of a linear displacement) of the linear position and the alignment of the x-ray tube 2 as well as its operating parameters ensues using control signals S that are generated by the control and evaluation device 14. The mammography apparatus can be controlled with the aid of input elements (symbolically illustrated by a keyboard 16 in the example) and different operating modes can be selected and implemented by the operating personnel.

During the acquisition of the multiple 2D x-ray images (individual images or projection data P_{α}) required for such a tomosynthetic 3D x-ray image T, the patient is in an uncomfortable and necessarily also painful situation. In order to provide at least subjective relief for the patient, an optical display 20 is arranged in the range of vision of the patient, with which optical display 20 the progress of the procedure (in the shown example information about the progress of the procedure required to generate a tomosynthetic 3D image data set T—pivoting of the x-ray tube 2 into the desired angle positions j and respective generation of a 2D x-ray image) is indicated. In the example of FIG. 1, this is a progress bar 24 traveling from left to right in the direction of the arrow 22. The travel speed and the length of the remaining portion provide the patient with information about the progress of the procedure and the likely end. An acoustic signal emitter 26 is additionally provided with which the progress of the procedure is indicated by an acoustic signal, for example when the progress bar 25 jumps forward by an increment.

In the illustrated exemplary embodiment the optical display 20 is shown as a separate apparatus that can be placed in the field of vision of the patient by the operating personnel. In principle it is also possible to integrate the optical display into the device, for example in the screen surface of the monitor 18 provided for image reproduction.

Instead of the illustrated running or progress bar, other geometric representation forms are also conceivable, for example a progress ring, a progress circle or a progress triangle. The progress indication can also ensue using a number of light-emitting diodes arranged side by side. The progress of the medical procedure can likewise be visualized by numbers, for example between 0 and 100%, or by numeric specification of the remaining time.

A further possibility for the optical presentation of the progress of the procedure is shown in FIG. 2. There the optical display 20 for the progress is visualized by a degree of filling 20 of an area rendered on a monitor in the shape of a breast.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for supporting implementation of a medical procedure on a patient, comprising the steps of:
conducting a medical procedure by interacting with a patient; and
during said medical procedure, presenting a visual display to the patient within the patient's field of view that optically indicates progress of the procedure toward an end of the procedure.

2. A method as claimed in claim 1 comprising, in said display, visibly presenting an indication of a temporal progress of the procedure relative to a total duration of the procedure.

3. A method as claimed in claim 1 comprising, in said display, visibly indicating a remaining duration of the procedure.

4. A method as claimed in claim 1 comprising additionally providing an acoustical indication of said progress of said procedure.

5. A method as claimed in claim 1 comprising conducting said medical procedure by generating a plurality of 2D x-ray images at respectively different projection angles, from which a 3D image data set is reconstructed.

6. A method as claimed in claim 5 comprising reconstructing a tomosynthetic 3D image data set from said plurality of 2D x-ray images.

7. An apparatus for implementing a medical procedure on a patient, comprising:
a medical device that interacts with a patient to implement a medical procedure on a patient; and
an optical display within the patient's field of view; and
a display generator that generates and presents an optical indication at said display indicating progress of the procedure toward an end of the procedure.

8. An apparatus as claimed in claim 7 comprising an acoustic signal emitter that emits an acoustic signal indicating said progress.

9. An apparatus as claimed in claim 7 wherein said medical device is an x-ray imaging device that acquires a plurality of 2D x-ray images of the patient respectively from different projection angles, and an image computer that reconstructs a 3D image data set from said plurality of 2D x-ray images.

10. An apparatus as claimed in claim 9 wherein said medical device is a mammography device that generates a tomosynthetic 3D image data set from said plurality of 2D x-ray images.
